(19) United States
(12) Patent Application Publication
(10) Pub. No.: US 2018/0071130 A1
(43) Pub. Date: Mar. 15, 2018

(54) METHOD FOR STIMULATION OF ENDOGENOUS CRANIOFACIAL OSTEOGENESIS VIA STEM CELL ACTIVATION

(71) Applicant: Advanced Facialdontics LLC, East Islip, NY (US)

(72) Inventors: Scott A. SIMONETTI, St. James, NY (US); Theodore BELFOR, Catskill, NY (US)

(21) Appl. No.: 15/704,692

(22) Filed: Sep. 14, 2017

Related U.S. Application Data
(60) Provisional application No. 62/394,245, filed on Sep. 14, 2016.

(51) Int. Cl. A61F 5/058; A61B 6/03; A61B 6/00

(52) U.S. Cl. CPC A61F 5/05891; A61B 6/505; A61B 6/4085; A61B 6/032

(57) ABSTRACT

The present disclosure relates to methods for stimulation of craniofacial osteogenesis which includes the step of providing light intermittent cyclic tensil force to the craniofacial area of a patient. In an embodiment, the step of providing mechanical stress is achieved through the use of removable oral orthotic devices which utilize unilateral bite block technology.

### CROSS-REFERENCE TO RELATED APPLICATIONS

The present application claims the benefit of U.S. Provisional Patent Application No. 62/394,245 filed on Sep. 14, 2016, entitled “ENDOGENOUS OSTEOGENESIS OF MESENCHYMAL STEM CELLS VIA MECHANOTRANSDUCTION ON THE AGED SKULL” the entire content of which is incorporated by reference herein.

### TECHNICAL FIELD

The present disclosure relates to methods for stimulation of craniofacial osteogenesis which includes the step of providing light intermittent cyclic tensil force to the craniofacial area of a patient. In an embodiment, the step of providing mechanical stress is achieved through the use of removable oral orthotic devices which utilize unilateral bite block technology.

### BACKGROUND

Facial aging is a dynamic process involving the aging of the soft tissue and bony structures of the face. The shape, size and volume of the bony orbit, for example, have all been shown to change in aging subjects. Although orthodontic apparatuses, methods, and procedures are currently used to correct a variety of oral health issues their use in stimulation of craniofacial osteogenesis (new bone formation) in the facial regions of a patient have not been described.

### SUMMARY

The present disclosure relates to methods for stimulation of craniofacial osteogenesis in a patient which includes the step of providing light intermittent cyclic tensil force (“cyclic mechanical stress”) to the craniofacial area of a patient. The application of cyclic mechanical stress is believed to stimulate a variety of different cellular responses including, for example, stimulation of endogenous mesenchymal stem cells located within the craniofacial sutures and periodontal ligament to proliferate and differentiate into bone tissue (osteogenesis) rather than fat tissue (adipogenesis).

In an embodiment, the step of providing mechanical stress is achieved through the use of removable oral orthotic devices that utilize unilateral bite block technology, such as for example, that provided by Unilateral BiteBlock Technology™. The use of such orthotic devices when positioned in the individual's mouth provides the required mechanical stress for stimulation of osteogenesis resulting in new bone and cartilage growth which in turn leads to an improvement of craniofacial symmetry. In a particular embodiment, the methods disclosed herein can be used to stimulate new bone formation in the region of the orbital eye sockets in an aging patient leading to a rejuvenated appearance.

In one aspect, the orthotic device is used intermittently for less than 12 hours a day. For example, the device may be worn by the patient at night during sleep and removed during the day. In another aspect, the device is worn for a variety of different treatment durations depending on the disorder being treated and the amount of new bone growth required. In one embodiment, for treatment of bone loss due to aging the device is worn for a minimum of twelve months.

As an additional step, the generation of new bone growth in a patient can be monitored throughout the treatment protocol. For example, cone beam computed tomography (CBCT) can be used to generate three dimensional maps taken at baseline and then repeatedly throughout treatment. Such monitoring can be used to determine the duration of treatment. Before and after object maps can be registered in three dimensional space using bone threshold and bone landmarks to detect new craniofacial bone growth within the patient. Commercially available software can be used to visualize and evaluate individualized three dimensional bone growth. Such software which can be used to process the biomedical images to detect new bone growth includes, for example, Analyze Direct Software developed by the Mayo Clinic.

The methods provided herein can be used to generate new bone growth within the craniofacial area of the treated patient. In one aspect, the disclosed methods can be used for regeneration of bone around the bony eye sockets (orbits) thereby reversing the natural aging process of the bony orbit. Such regeneration of bone of the orbits can result in restoration of craniofacial symmetry leading to successful facial rejuvenation. The methods disclosed herein may also be used to treat osteoporosis in general and/or to stimulate bone growth in patients suffering from facial fractures or jaw osteonecrosis.

### BRIEF DESCRIPTION OF THE DRAWINGS

FIG. 1 is a view of an oral orthotic device (NightBlock™)

FIG. 2 is a view of an oral orthotic device (Preventive Oral Device (POD™)

FIG. 3 is a view of as oral orthotic device (OrthoBlock™).

FIG. 4A is a computerized tomographic scan of a young male patient.

FIG. 4B is a computerized tomographic scan of an old male patient;

FIG. 5A is a photograph of a treated patient demonstrating restoration of facial symmetry.

FIG. 5B is a computerized tomographic scan of a patient before and after treatment.

FIG. 5C is a depiction of new bone volume in a treated patient.

FIG. 6 is a color histogram demonstrating new bone growth in a treated patient.

FIG. 7A is a computerized tomographic scan of a patient before and after treatment.

FIG. 7B is a depiction of new bone volume in a treated patient.

FIG. 7C is a color histogram demonstrating new bone growth in a treated patient.

FIG. 8A is a computerized tomographic scan of a patient before and after treatment.

FIG. 8B is an object map showing areas of bone growth (left) and volume changes in bone and soft tissue.

FIG. 8C is a depiction of new bone volume in a treated patient.

FIG. 9 is a depiction of new bone growth in a treated patient.

### DETAILED DESCRIPTION

The accompanying drawings, which are incorporated in and constitute a part of the specification, illustrate embodiments of the disclosure and, together with a general description of the disclosure given above, and the detailed description of the embodiment(s) given below, serve to explain the principles of the disclosure.

Embodiments of the present disclosure are described in detail with reference to the drawings.

As used herein, the term “clinician” refers to a doctor, nurse, or other care provider and may include support personnel. The term “patient” refers to an individual being treated by a clinician. As used herein, the term osteogenesis refers to the formation/development of new bone.

The present disclosure relates to methods for stimulation of craniofacial osteogenesis in a patient which includes the step of providing light intermittent cyclic tensil force (“cyclic mechanical stress”) to the craniofacial area of a patient. The application of cyclic mechanical stress as disclosed herein can be used to stimulate a variety of different cellular responses including, for example, stimulation of stem cells including endogenous mesenchymal stem cells located within the craniofacial sutures and periodontal ligament to proliferate and differentiate into bone tissue (osteogenesis) rather than fat tissue (adipogenesis). In one aspect, the use of oral orthotic devices as described herein are designed, for example, to increase bone formation, remodel the mandibular condyle, realign the mandible, increase bone deposition around the boney orbits, widening of the sphenoid, and increase bone deposition at the maxillary zygoma. Further, given that an increase in adipogenesis of mesenchymal stem cells can lead to osteoporosis, the methods disclosed herein may be used to inhibit, or treat, osteoporosis. In a specific aspect, the methods can be used to treat or inhibit osteoporosis in the craniofacial region of the patient.

In an embodiment, the step of providing mechanical stress is achieved through the use of removable oral orthotic devices that utilize unilateral bite block technology. The use of such orthotic devices when positioned in the individual's mouth will provide the required mechanical stress for stimulation of craniofacial osteogenesis resulting in new bone and cartilage growth which in turn leads to an improvement of craniofacial symmetry. The method includes providing an oral orthotic device to a patient and applying pressure to the orthotic device to cause mechanical stress to the cranium of the patient.

In one aspect, the exogenous mechanical stress to be exerted in the craniofacial region has one or more of the following features including magnitude, direction, frequency, point of application, and duration. Any cyclic strain stimulating oral orthotic device designed for endogenous mesenchymal stem cell stimulation, would achieve one or more of the force properties and provide both tensile and compressive forces. The ideal orthotic device should provide a magnitude of force much lower than maximum physiologically attainable magnitudes (light force versus heavy force) to the sutures and PDL. The device should also apply the force in a physiological direction (in the direction of normal mastication). Also, the frequency should be as high as possible. With regard to frequency, a static force has less stimulatory effect than higher frequency cyclic forces. Accordingly, the application of brief doses of cyclic force induces osteogenesis more effectively than static forces. Next, the device should apply force at the most physiological point (the occlusal surfaces accustomed to maximal forces). Lastly, the device should apply strain for about 4-12 hours a day. The device should be comfortable and cause no harm. Therefore, under optimal cellular conditions, by applying the proper magnitude of light cyclic mechanical force to the mesenchymal stem cells within the cranial sutures and PDL, in a physiological direction and point, with high frequency and duration of between 4-12 hours, one can expect to stimulate bone growth pathways in addition to slowing age related osteoporosis.

In an embodiment of the invention, publically available orthotic devices may be used to generate the mechanical stress required for new bone growth. Specific devices that may be used to generate mechanical strain include, for example, Upper and Lower HomeoBlock™ Appliances, Unilateral BiteBlock Technology and POD (preventive oral device). Detailed descriptions of the devices, their construction and use for generation of mechanical stress to the cranium of the patient is described in PCT/US2017/028593 which claims the benefit of U.S. Provisional Patent Application No. 62/325,176, filed on Apr. 20, 2016, entitled “PREVENTIVE ORAL DEVICE (POD)”, U.S. Provisional Patent Application No. 62/325,192, filed on Apr. 20, 2016, entitled “RETAINER APPLIANCE”, and U.S. Provisional Patent Application No. 62/325,185, filed on Apr. 20, 2016, entitled “ORAL ORTHOTIC APPLIANCE (NIGHTBLOCK)”, the entire contents of each of which are incorporated by reference herein. Additional devices and methods are disclosed in U.S. Pat. Nos. 7,357,635 and 7,314,372 to Belfor et al. the contents of which are incorporated herein by reference. It is well within the skill of the attending clinician to design a force pattern that includes the required magnitude, direction, frequency, point of application to be applied by the device to achieve the desired results.

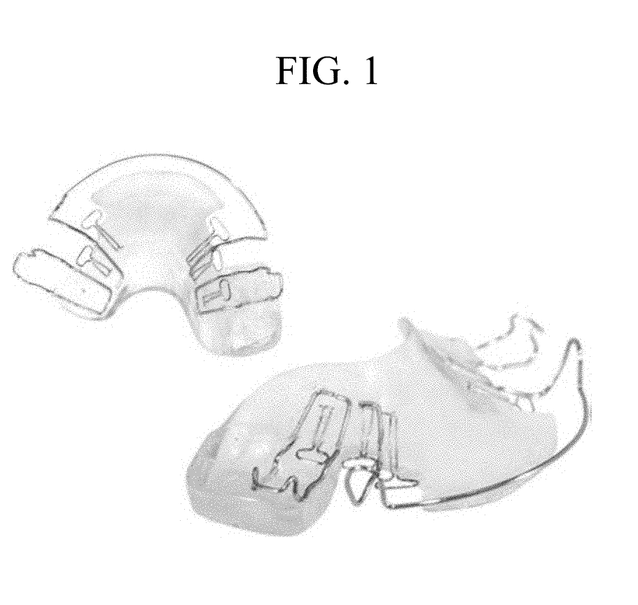

In one aspect, a NightBlock™ appliance (FIG. 1) may be used to provide mechanical stress to the craniofacial region of a patient. The appliance is a custom made oral orthotic worn during the night on the maxillary arch. It acts as a mandibular repositioner and is designed to protect the teeth from damaging parafunctional occlusal forces such as clenching and grinding. When worn, it is designed to reduce bite forces and reposition the mandibular condyles which thereby decompresses the articular disks. The NightBlock™ appliance corresponds to FIGS. 10-12 of PCT/US2017/028593.

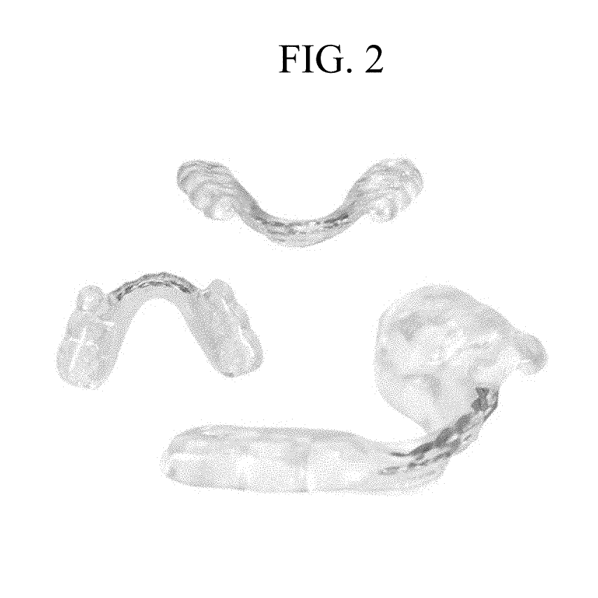

In another aspect, a “Preventive Oral Device” or POD™ (FIG. 2) is used to provide the required mechanical stress to the craniofacial region of a patient. A POD is a mandibular splint designed to reduce stress and can be used as a daytime or night time appliance. Biting on the unilateral bite block and allows the tongue to move forward while the bite block decompresses the jaw joint. Combined with controlled breathing and used for 15-20 minutes at a time during the day, a reduction of cortisol and improved autonomic nervous system balance has been demonstrated. The POD™ (Preventive Oral Device) is designed to improve a patient's jaw muscle function and protect the TMJ to lesson symptoms such as jaw clenching, tension headaches, neck, shoulder and jaw pain. It can be worn at night time or during the day. The design creates more space for the tongue to move up towards the palate and out of the throat increasing the size of the airway. The POD™ appliance corresponds to FIGS. 1-7 of PCT/US2017/028593.

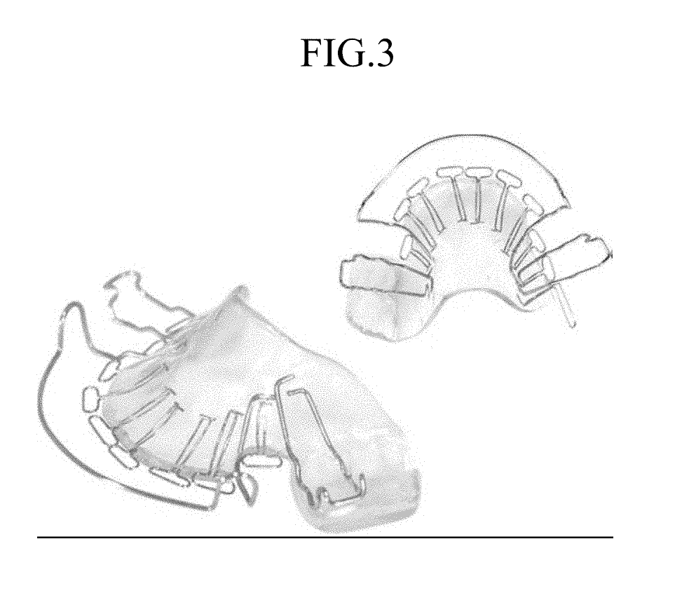

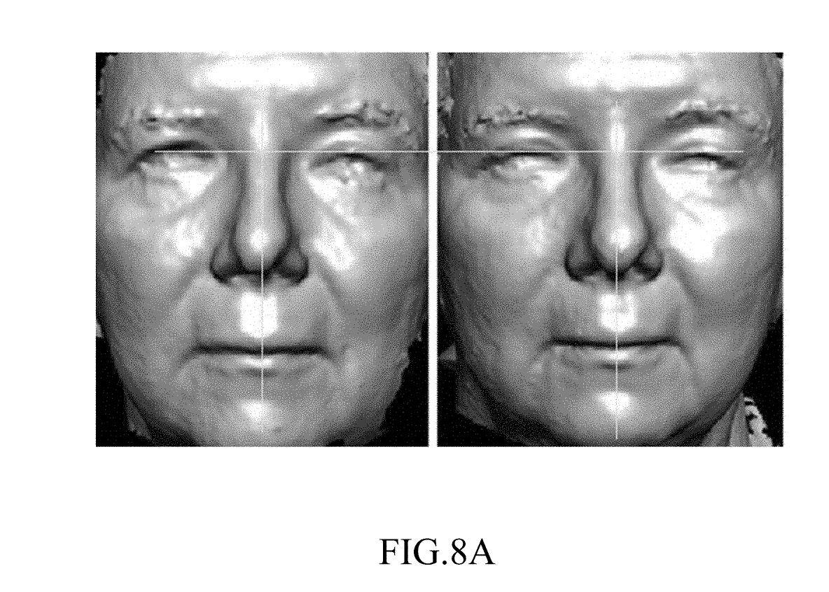

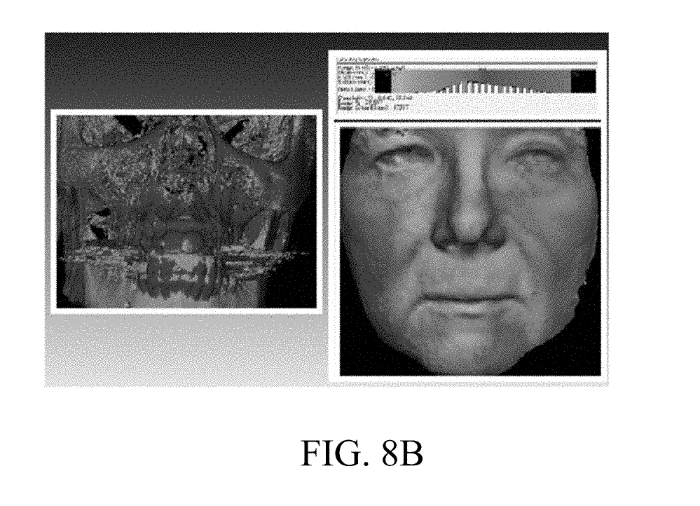

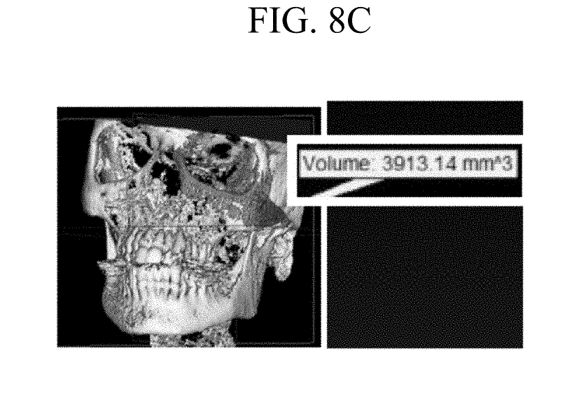

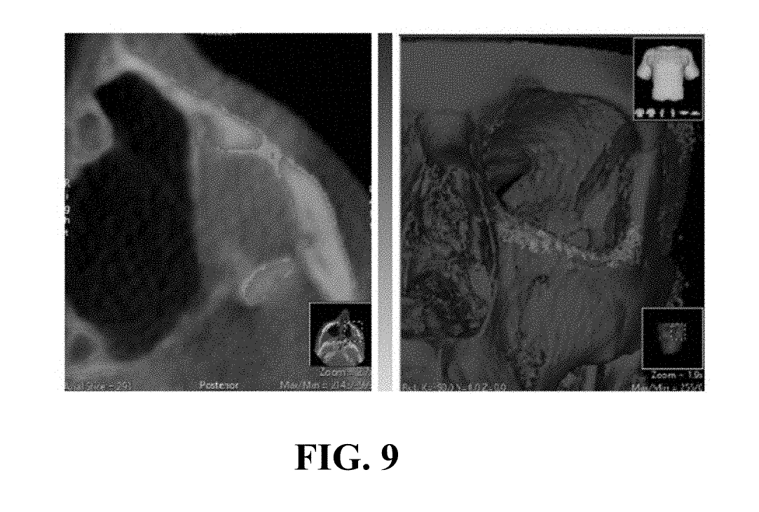

In yet another aspect, an OrthoBlock™ appliance (FIG. 3) may be used to provide the required mechanical stress. The OrthoBlock™ appliance is a custom made oral orthotic worn during the night on the maxillary arch or mandibular arch. It acts as a mandibular repositioner and is designed to protect the teeth from damaging parafunctional occlusal forces such as clenching and grinding. When worn, it is designed to reduce bite forces and reposition the mandibular condyles which thereby decompresses the articular disks. It can be worn as an orthodontic retainer and can also be used as a functional orthodontic appliance for limited tooth movement. The OrthoBlock™ appliance corresponds to FIGS. 8-9 of PCT/US2017/028593.

The orthotic device may be inserted into a patient's oral cavity and retained in the mouth for varying periods of time. In one aspect of the invention, the orthotic device is used intermittently for less than a twenty four hour time span. In an embodiment, the orthotic device is used for less than 12 hours a day. In another embodiment, the orthotic device is used for between 8 to 12 hours a day. In yet another embodiment, the orthotic device is used between 6 to 12 hours a day. For example, the device may be worn by the patient at night during sleep and removed during the day.

In another aspect, the device is worn intermittently on a daily basis for a variety of different treatment durations depending on the disorder being treated. For example, the device can be worn daily intermittently for a minimum of three months. In an embodiment, for treatment of bone loss due to aging, the device is worn for a minimum of twelve months. In one aspect, the generation of new bone growth can be monitored throughout treatment and the duration of treatment can be adjusted accordingly to attain the desired result.

As an additional step, the generation of new bone growth in a patient can be monitored throughout the treatment protocol In one aspect of the invention, scans may be performed every 4 to 8 months following commencement of treatment to detect of new bone formation, with successive scans compared to the baseline scan obtained prior to commencement of treatment. In one aspect, the scans are performed every 6 months following commencement of treatment. A number of methods well known to those of skill in the art may be used to monitor the generation of new bone growth including, for example, cone beam computed tomography (CBCT), micro-computed tomography (micro-CT), high resolution MRI or digital stereophotogrammetry. For example, before and after object maps can be registered in three dimensional space using bone threshold and bone landmarks to detect new craniofacial bone growth within the patient. Commercially available software, such as AnalyzeDirect (provided by the Mayo Clinic), can be used to process the biomedical images to detect such new bone growth. See, Figure

In a specific embodiment of the invention, utilizing AnalyseDirect software, assessment of bone differences are detected through comparisons of pre- and post-treatment CBCT scans. Patient post-treatment scans are registered to pre-treatment scans. The transformed post-treatment scan and fused maps are saved. The transformed post-treatment scans and difference maps are then used as an input for segmentation. Using threshold segmentation and then region growing, difference regions are segmented from into objects from the area of interest and the resulting object map is saved. Volumes of the difference regions are then calculated to detect new bone growth.

The methods disclosed herein can be used to stimulate proliferation of stem cells including mesenchymal stem cells within the cranial facial suture system of the patient. Such mesenchymal stem cells may differentiate within the craniofacial to form new bone tissue within the area. Alternatively, the stimulated mesenchymal stem cells can migrate from the cranial facial area of the patient and exert a therapeutic benefit to other regions of the patient's body.

In one aspect, the disclosed methods can be used for regeneration of bone around the bony eye sockets (orbits) of a patient thereby reversing the natural aging process of the bony orbit. Such regeneration of bone of the orbits can result in restoration of craniofacial symmetry leading to successful facial rejuvenation. The disclosed methods as described herein may also serve as a useful adjunctive treatment when combined with plastic surgery procedures such as soft tissue techniques like blepharoplasty and fat transfer around the orbit. Traditional plastic surgery techniques typically aim to address thinning loose skin, weakening of ligaments, and atrophy of fat and tissue descent due to gravity. However, these techniques may not be successful in camouflaging the bony changes of the orbit. Accordingly, the presently disclosed methods may be used to stimulate bone deposition at the periorbital with an associated improvement in eyelid ptosis and lid position.

The methods disclosed herein may also be used to stimulate bone growth in patients suffering from facial fractures. Facial fractures include broken bones anywhere on the face such as the nose, cheekbones, the area around the eyes, and the upper and lower jaw. Such facial fractures may result from trauma to the face from motor vehicle crashes, sporting injuries, falls and cosmetic surgery. In one aspect, the bones in the face may fracture due to weakened by a dental procedure or condition.

The methods disclosed herein may also be used to treat osteonecrosis of the jaw. Osteonecrosis of the jaw may develop when the jawbone fails to heal after a minor injury, such as getting a tooth pulled. Additionally, bisphosphonates that are used to treat post-menopausal osteoporosis have been linked to osteonecrosis of the jaw. In addition, bisphosphonates are also used to treat cancer that has spread to the bone and the risk of osteonecrosis of the jaw is much greater for people taking high doses of these drugs to treat cancer. Accordingly, the methods disclosed herein, designed to stimulate osteogenesis in the facial region of a patient, may be used to treat osteonecrosis of the jaw.

The use of oral orthotic devices as disclosed herein, is considered to create an epigenetic response, mediated by the removal of previous epigenetic tags that created the phenotype of craniofacial asymmetry and systemic inflammation, or the addition of new epigenetic tags that transcribed the proper genes that enabled improved symmetry and osteogenesis and improve breathing by reshaping the hard palate, even in aging patients. In one aspect, insertion of the orthotic device creates the proper cyclic intermittent light strain, coupled with a reduction in reactive oxygen species by improving breathing and nitric oxide homeostasis, to enable endogenous stem cell activation and osteogenesis in the craniofacial region of a patient.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

### EXAMPLES

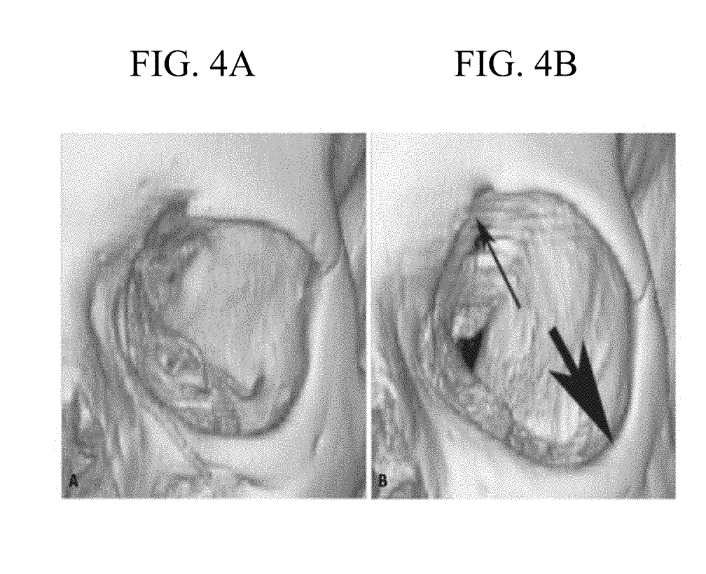

Facial aging is a dynamic process involving the aging of the soft tissue and bony structures of the face. The shape, size and volume of the bony orbit have all been shown to change with age in both male and female subjects (FIG. 4). Presented are four case studies illustrating that wearing a dental oral appliance at night, which is designed to send appropriate signaling to the cranial sutures and facial bones, can result in additional bone growth and/or bone remodeling that opposes the normal aging process of bone. As disclosed, the use of an orthopedic/orthodontic devices (featuring Unilateral BiteBlock Technology™) that is worn only at night, results in remodeling of the bone around the orbit and reverses the natural aging process of the bony orbit.

### Materials and Methods

A cone beam computerized tomographic scan (CBCT) scan and three dimensional facial scan, (3dMD Face) were taken for each patient. Upper and lower dental models were fabricated to establish a baseline. Homeoblock dental appliances were fabricated for the patients. (See, U.S. Pat. No. 7,357635 and U.S. Pat. No. 7,314,372 for a description of homeoblock dental applicances as well as their use). Although, the examples disclosed below relate to the use of homeoblock devices, it is understood that any oral orthotic device that provides cyclic mechanical stress to the craniofacial region of the patient may be used for stimulation of osteogenesis. The Homeoblock™ utilizes an orthodontic expansion screw along with our Unilateral BiteBlock Technology™. The unilateral 5 mm bite block is placed on the second bicuspid and first molar on the less developed side, which is the side with a deeper naso-labial depression or lower eye, thinner upper lip or deeper pre jowl region. The appliances were worn for a maximum of 10-12 hours each day (mainly during sleep) for a minimum of 12 months. The patients were instructed to advance the expansion screw 0.125 mm (¼ turn) each week. As disclosed below, the before and after object maps are registered in three dimensional space using the bone threshold and hundreds of bone landmarks. (“Analyze 10.0” developed by the Mayo Clinic). Where the red shows through is where the additional bone has been generated.

### Results

### Case Study I

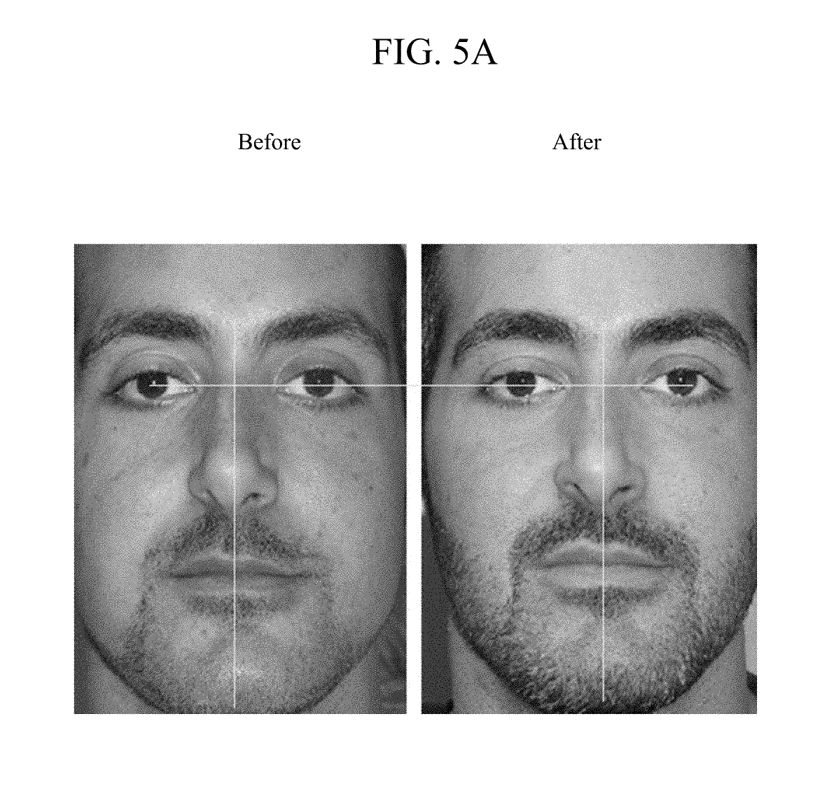

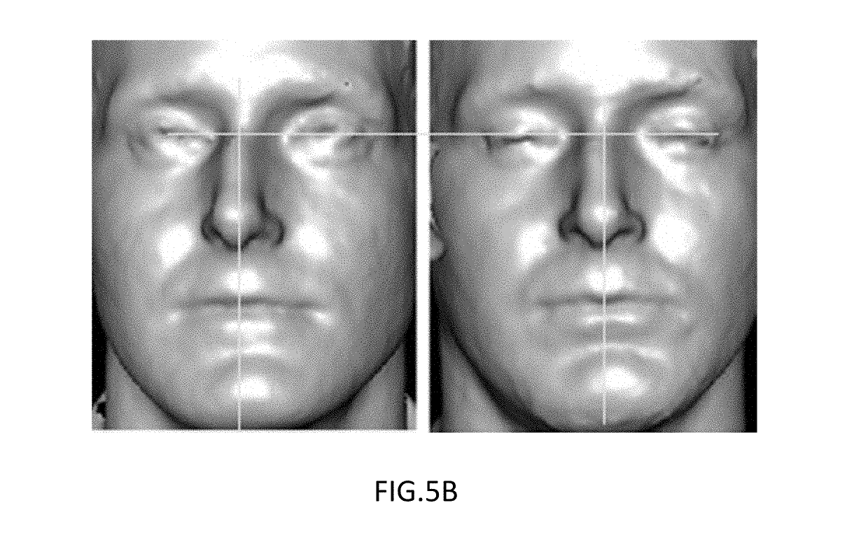

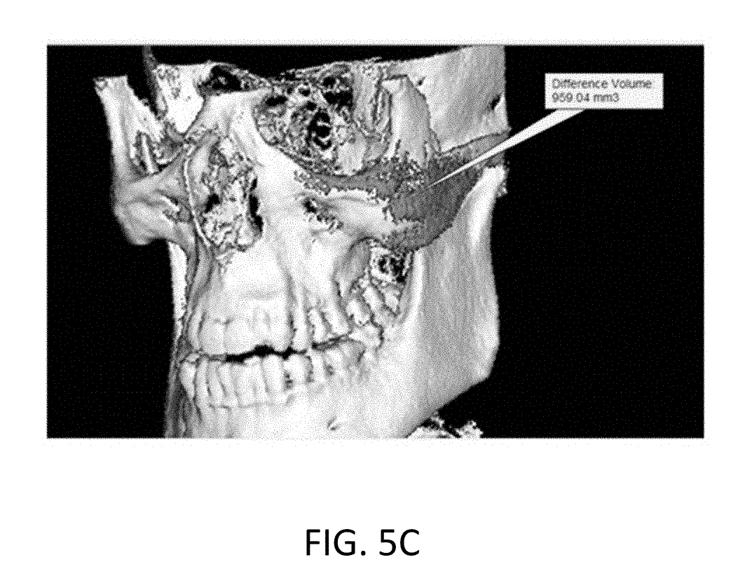

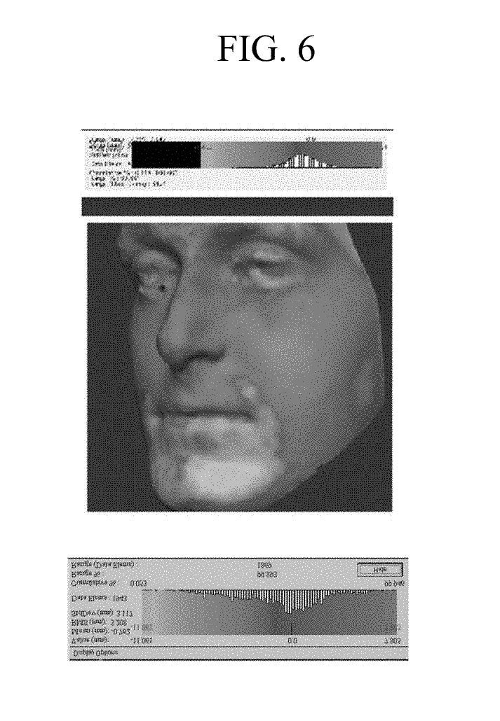

A healthy 40 year old male before and after 2 years appliance therapy treatment shows a reduction in the ptosis of the upper and lower eyelids and improved facial bone structure (FIG. 5A). Using digital stereophotogrammetry with an eight camera system an accurate three dimensional object was created (FIG. 5B). Using multiple landmarks a before and after image was evaluated for volume change. The red area around the orbit and the zygomatic arch shows increased volume and helps to confirm the new bone growth indicated (3DMD Face) (FIG. 6).⁸

The transformed post-treatment scan and fused difference map were saved. The transformed post-treatment scans and difference maps were used as an input for segmentation. Using threshold based segmentation and then “region grow”, difference regions were segmented into objects from the area of interest and the resulting object map saved. Volumes of the difference regions were then calculated (FIG. 5C). A difference volume of 959 mm³ was detected.

### Case Study II

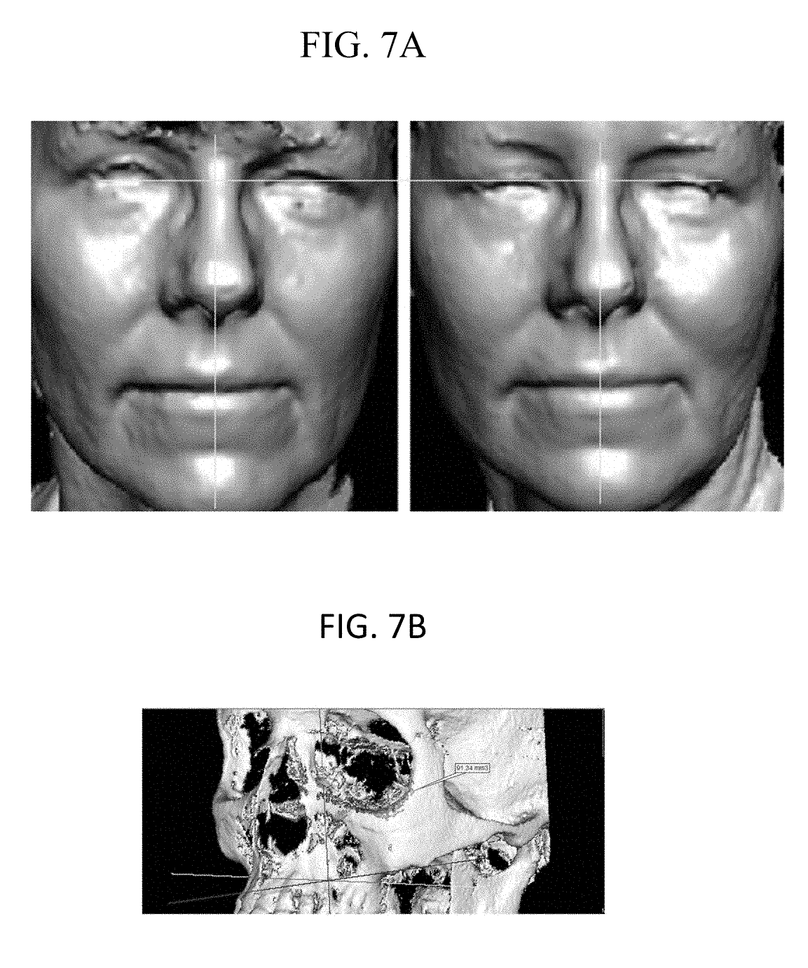

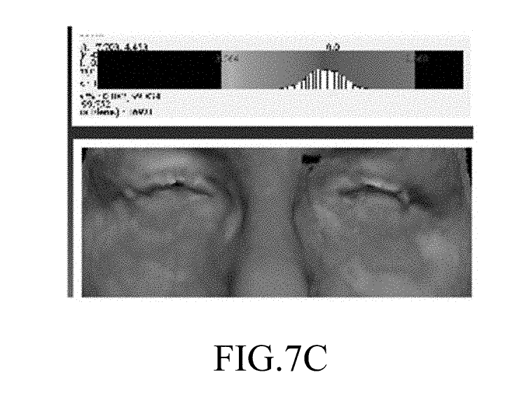

A healthy 50 year old female was treated for 2 years with appliance therapy. Before and after 2 years appliance therapy results are depicted in FIG. 7A. Red area around the left orbit confirms pattern of bone growth (3dMD Face) (FIG. 7C). The patient appears to show more level eyes along with improved facial symmetry. Analysis using region grow Analyze 10.0 shows additional voxel volume at the inferior border of the orbit 91.34 mm³ (FIG. 7B).

### Case Study III

A healthy 60 year old female was treated for two years with dentofacial appliance therapy. The object map on the left shows the areas of bone growth in red (FIG. 8A). The photo on the right shows volume changes in bone and soft tissue (FIG. 8B). Two different software applications support the same results. This patient shows 3,913 mm³ increased bone volume around the left orbit (FIG. 8C).

### Case Study IV

The present disclosure describes a Case Study of a 69 year old male with an extensive medical history, who wore nighttime only custom oral orthotics (Upper and Lower HomeoBlock™ Appliances) for 18 months. Treatment also included nighttime Buteyko mouth tape and nasal tape strips. There were no other reported changes in diet, or exercise routine during the treatment. The patient reported the following improvements in general health after 18 months of orthotic treatment: 40 pound weight loss, improvement in his poor posture, TMD resolved, GERD eliminated, two reductions in antihypertensive medications, no longer necessary to take a diuretic or antidepressant, and his autoimmune diseases of forty year duration resolved (Crohn's and Chronic Skin eruptions-psoriasis and pyoderma gangrenosa, and Shaumberg's Purpura).

In addition to these patient reported outcomes, the case study includes before and after CBCT scans along with Analyze™ software designed by the Mayo Clinic, that show skeletal changes after treatment. These changes include remodeling of the mandibular condyle, realignment of the mandible, bone deposition around the boney orbits, widening of the sphenoid, bone deposition at the maxillary zygoma, a reshaping of the maxillary hard palate, remodeling of the maxillary teeth and alveolar arch, remodeling of the mandibular teeth and alveolar arch, and a widening of the pharyngeal airway. Visually, the before and after treatment show dramatic changes that correlate with the CBCT changes.

The four case studies presented herein demonstrate cranial facial osteogenesis in the orbital region using data acquired from CBCT scans of patients wearing a dentofacial orthopedic/orthodontic oral appliance at night time. In addition, FIG. 9 is a representation of an additionally treated patient demonstrating the growth of new bone following use of the methods disclosed herein. New bone growth is depicted in green.

What is claimed is:

1. A method of stimulating endogenous craniofacial osteogenesis, the method comprising:
providing an orthotic device to a patient;
positioning the orthotic device in a mouth of the patient to provide mechanical stress in the craniofacial region for at least a predetermined period of time for endogenous craniofacial osteogensis to take place; and
removing the device after the device has been in said patients mouth for all of the at least predetermined period of time.

2. The method of claim 1 further comprising:
monitoring the development of new bone growth in the craniofacial region throughout treatment wherein facial scans obtained before commencement of treatment are compared to scans obtained after commencement of treatment.

3. The method of claim 2 wherein the facial scan is a cone beam computerized tomographic scan.

4. The method of claim 1, wherein stimulation of craniofacial osteogensis is in the region of the bony eye orbits.

5. The method of claim 4, wherein the stimulation of craniofacial osteogenesis results in periobital rejuvenation.

6. The method of claim 1, wherein the device is of FIG. 1.

7. The method of claim 1, wherein the device is the device of FIG. 2.

8. The method of claim 1, wherein the device is the device of FIG. 3.

9. A method of stimulating mesenchymal stem cells within cranial sutres, the method comprising:
providing an orthotic device to a patient;
positioning the orthotic device in a mouth of the patient to provide mechanical stress in the craniofacial region for at least a predetermined period of time for endogenous craniofacial osteogensis to take place; and
removing the device after the device has been in said patients mouth for all of the at least predetermined period of time.

10. The method of claim 9 further comprising:
monitoring the stimulation of mesenchymal stem cells, which in turns leads to development of new bone growth in the craniofacial region, throughout treatment wherein facial scans obtained before commencement of treatment are compared to facial scans obtained after commencement of treatment.

11. The method of claim 10, wherein the facial scan is a cone beam computerized computerized tomographic scan.

12. The method of claim 9, wherein the stimulation of mesenchymal stem cells results in craniofacial osteogensis is in the region of the bony eye orbits.

13. The method of claim 12, wherein the stimulation of craniofacial osteogenesis results in periobital rejuvenation.

14. The method of claim 1, wherein said method is used in in combination with blepharoplasty.

15. The method of claim 1, wherein said method is used in combination with fat transfer around the orbit.

16. The method of claim 9, wherein the device is the device of FIG. 1.

17. The method of claim 9, wherein the device is the device of FIG. 2.

18. The method of claim 9, wherein the device is the device of FIG. 3.
